(19) United States
(12) Patent Application Publication
(10) Pub. No.: US 2020/0016078 A1
(43) Pub. Date: Jan. 16, 2020

(54) COMPOSITION FOR TRANSDERMAL DELIVERY OF MICRONUTRIENTS AND METHOD OF PREPARATION THEREOF

(71) Applicant: CERELIA NUTRITECH PVT LTD, Buvvanapalli, Andhra Pradesh (IN)

(72) Inventors: Ruchi SINGH, Mumbai (IN); NVV Kiran VUPPALA, Buvvanapalli (IN); Nikhil MUPPAVARAPU, Hyderabad (IN); Dipti Sundar MOHANTY, Bhubaneshwar (IN)

(21) Appl. No.: 16/469,162

(22) PCT Filed: Mar. 8, 2018
(86) PCT No.: PCT/IN2018/050133

§ 371 Date: Jun. 13, 2019

(30) Foreign Application Priority Data
Mar. 8, 2017 (IN) 201741008174

(51) Int. Cl. A61K 9/127; A61K 9/00; A61K 9/06; A61K 31/714; A61K 31/519; A61K 33/26; A61K 47/10

(52) U.S. Cl. CPC A61K 9/1272; A61K 9/0014; A61K 9/1277; A61K 47/10; A61K 31/714; A61K 31/519; A61K 33/26; A61K 9/06

(57) ABSTRACT

The present invention provides micronutrients loaded lipid vesicles and its process of preparing. The micronutrient loaded lipid vesicles preferably phospholipid vesicles comprise of micronutrients, lipid preferably phospholipid and edge activators. The present invention further covers compositions incorporating such lipid vesicles and its process of preparing. The compositions include transdermal compositions such as i) peel-off gel, ii) film forming gel compositions, iii) lotions, iv) face cream composition, v) clear transparent gel, vi) patch etc. The micronutrients loaded vesicles and the compositions thereof such that they provide rapid and improved permeation of micronutrients through skin into blood.

### FIELD OF THE INVENTION

The present invention relates to micronutrients loaded lipid vesicles/lecithin vesicles and further compositions incorporating such lipid vesicles/lecithin vesicles. Lipid vesicles are preferably phospholipid vesicles/lecithin vesicles particularly lecithin Vesicles and compositions include transdermal i) peel-off gel composition comprising micronutrients loaded lipid vesicles/lecithin vesicles; ii) film forming gel composition comprising micronutrients loaded lipid vesicles/lecithin vesicles; iii) lotion composition comprising micronutrients loaded lipid vesicles/lecithin vesicles; iv) face cream composition comprising micronutrients loaded lipid vesicles/lecithin vesicles; v) clear transparent gel composition comprising micronutrients loaded lipid vesicles/lecithin vesicles; v) patch composition comprising micronutrients loaded lipid vesicles/lecithin vesicles. The lipid vesicles/lecithin vesicles comprise of lecithin and optionally edge activators for delivery of hydrophilic as well as lipophilic micronutrients.

### BACKGROUND OF THE INVENTION

There are several forms of compositions for delivery of micronutrients. These include oral, parenteral & intranasal forms of delivery route. Even though the oral route is the most common route for delivery it is associated with several limitations. The oral bioavailability of these bioactives is limited because of physicochemical and physiological events that occur within the gastrointestinal tract (GIT) after their ingestion. When a dietary supplement is swallowed, it is digested by stomach acids and then metabolized by the liver to the extent that only a small portion of the original dosage enters the bloodstream. Thus, the liver dramatically reduces the bioavailability of oral supplements. In pregnant women maternal metabolism is altered by hormones that mediate the redirecting of nutrients to the placenta and mammary gland as well as the transfer of nutrients to the developing infant. Absorption of some vitamins is dependent on presence of intrinsic factors; oral bioavailability is majorly affected due to deficiency of these intrinsic factors. To overcome these problems, a transdermal system is being developed which will improve the efficacy and hence patient compliance.

Even though transdermal route is a very promising approach for delivery of nutrients, drugs, etc, skin is a very formidable barrier to entry of both small and large molecules. Skin permeability depends upon the physical properties of drugs or micronutrients which facilitates its passive diffusion through skin. For therapeutic quantities to permeate through the skin, the barrier properties of the Stratum corneum must be overcome. Because of the selective nature of the skin barrier, only few selected sections of drugs can be delivered to the skin for systemic action at therapeutic levels. A lipophilic drug, can cross the Stratum corneum, but once it enters the more aqueous lower regions of the epidermis the rate of diffusion decreases. Thus, as the diffusion of a very hydrophobic molecule proceeds into deeper layers of the skin, diffusion slows, and the concentration gradient (from Stratum corneum down to the viable tissue) falls. Similarly, for a water soluble drug, lipophilic stratum corneum is the main barrier which limits their use for transdermal delivery.

Lecithin being Amphiphilic in nature can encapsulate both hydrophilic as well as lipophilic molecules and improve their Permeation. Lipid preferably phospholipids are recognized as penetration enhancers because the absorption of lipid preferably phospholipids on the skin can increase tissue hydration, consequently increasing drug permeation.

When lipid preferably phospholipids are applied to the skin as delivery vehicles, they can fuse with stratum corneum lipids, perturb its structure and facilitate drug delivery. Therefore, lecithin is the ideal surfactant for preparing pharmaceutically acceptable systems. In order to effectively deliver the micronutrient loaded lecithin vesicles transdermally, the micronutrient loaded lecithin vesicles will be loaded in a transdermal vehicle i.e. film forming gel.

Transdermal formulations, such as ointments, are oily and gritty thus making the formulation less acceptable for patients. A sufficient concentration of a transdermally applied therapeutic agent must be loaded into the vehicle to ensure an adequate concentration gradient between the formulation and the skin, in order to attain adequate release of the drug into the skin. Transdermal patches do not possess the capability to release the entire amount of the drug incorporated into the skin, and huge quantities of drug are wasted once the patch is peeled off from the skin. Hence what is desired is a combination of aesthetically and cosmetically appealing system capable of delivering required quantity of drug into the skin without having the disadvantages of a conventional patch.

### OBJECT OF THE INVENTION

One object of the present invention is to provide micronutrients loaded lipid vesicles, preferably phospholipid vesicles/lecithin vesicles particularly lecithin vesicles. The lecithin vesicles comprise of lecithin and optionally edge activator for delivery of hydrophilic as well as lipophilic micronutrients.

Second object of the present invention is to provide various further compositions comprising micronutrients loaded lipid vesicles preferably phospholipid vesicles/lecithin vesicles particularly lecithin vesicles. These compositions include transdermal compositions such as i) peel-off gel, ii) film forming gel compositions, iii) lotions, iv) face cream composition v) clear transparent gel compositions vi) patch etc.

The third object of the present invention is to provide micronutrients loaded vesicles and the compositions thereof such that they provide rapid and improved permeation of micronutrients through skin into blood. By modifying the contents of vesicles and their further compositions, various types of release profiles can be achieved.

Fourth object of the present invention is to provide process of preparing micronutrients loaded lipid vesicles/lecithin vesicles preferably phospholipid vesicles/lecithin vesicles and further processes to prepare their transdermal compositions that provide rapid and improved permeation through skin into blood.

Fifth object of the present invention is to prepare various transdermal compositions of micronutrients for treatment/prevention of anemia.

### SUMMARY OF THE INVENTION

First aspect of the present invention is to provide micronutrients loaded lipid vesicles/lecithin vesicles. The lecithin vesicles comprise of lecithin and optionally edge activator for delivery of hydrophilic as well as lipophilic micronutrients.

Second aspect of the present invention is to provide various compositions comprising micronutrients loaded lipid vesicles/lecithin vesicles preferably phospholipid vesicles/lecithin vesicles particularly lecithin Vesicles. These compositions include transdermal compositions such as i) peel-off gel, ii) film forming gel compositions, iii) lotions, iv) face cream composition v) clear transparent gel compositions vi) patch etc.

The third aspect of the present invention is to provide micronutrients loaded vesicles and the compositions thereof such that they provide rapid and improved permeation of micronutrients through skin. By modifying the contents of vesicles and their further compositions, various types of release profiles can be achieved.

Fourth aspect of the present invention is to provide process of preparing micronutrients loaded lipid vesicles/lecithin vesicles preferably phospholipid vesicles/lecithin vesicles and further processes to prepare their transdermal compositions that provide rapid and improved permeation through skin into blood.

Fifth aspect of the present invention is to prepare various transdermal compositions of micronutrients for treatment/prevention of anemia.

### BRIEF DESCRIPTION OF DRAWINGS

FIG. 1: Contour plot for particle size.

FIG. 2: Contour plot for Poly dispersibility index.

FIG. 3: Contour plot for % Cumulative Release at the end of 24 hrs.

FIG. 4A: Comparative diffusion of vitamin B12 loaded lecithin vesicles prepared as described by example 5A and Vitamin B12 solution in water.

FIG. 4B: Comparative diffusion of folic acid loaded lecithin vesicles prepared as described by example 5C and plain folic acid.

FIG. 5: In vivo release profile of reference formulation by oral route and test formulation by transdermal route.

FIG. 6: Size distribution data of Vitamin B12 loaded lecithin vesicles

FIG. 7: Size distribution data of folic acid loaded lecithin vesicles.

FIG. 8: Size distribution data of ferrous sulphate loaded lecithin vesicles.

### DETAILED DESCRIPTION

Many patents and research articles report topical application of micronutrients however they do not provide any data to substantiate transdermal uses of the compositions disclosed therein. Mere topical application using any composition system wherein lipid micro or nano particles are used is not sufficient. One should be able to substantiate data using at least one animal model. Godin et alⁱ reports as follows: “Among rodents, rat skin has more structural similarities to human tissue (Table 1) . . . Regarding the rat skin, permeation kinetic parameters are frequently comparable with human skin.”

Further, he refers to a study by Harada et alⁱⁱ which suggests that the Wistar rat and nude mouse performed similarly to human cheek, neck, and inguinal skin.

In the present invention, inventors have successfully developed micronutrients loaded lipid vesicles/lecithin vesicles preferably lecithin vesicles and their further compositions which can be applied topically allowing gradual, continuous absorption of micronutrients in body. They have then compared the further compositions comprising micronutrients loaded lecithin vesicles with the oral delivery of micronutrients to substantiate suitability of the further compositions by transdermal delivery.

In a first aspect, the present invention provides at least one micronutrient loaded lipid vesicles/lecithin vesicles. The phospholipids such as lecithin are amphiphilic in nature. Such lipid vesicles can encapsulate both hydrophilic as well as lipophilic micronutrients/drug.

The lecithin vesicles comprise of lecithin and optionally edge activator for delivery of hydrophilic as well as lipophilic micronutrients. The ratio of micronutrient to lecithin is from 1:0.5 to 1:5, more preferably from 1:0.5 to 1:2 and most preferably from 1:1 to 1:2.

In an embodiment as presented under example 5A, a ratio of 1:1 is used. In another embodiment as presented under example 1 a ratio of 1:2 is used.

Micronutrients without limitations include one or more of Vitamin-A, Vitamin-D, Vitamin-E, Vitamin-C, Vitamin-B1, Vitamin-B2, Vitamin-B3, Vitamin-B6, Vitamin-B9, Vitamin-B12, Iron, Zinc, Copper, Selenium, Iodine and wherever applicable their salt forms.

In one embodiment, micronutrient is Vitamin B12 as exemplified in examples 1 and 5A. In another embodiment, micronutrient is folic acid as exemplified in example 5C. In yet another embodiment, micronutrient is an iron salt (ferrous sulphate) as exemplified in example 6. In other embodiments, iron salt such as pyrophosphate or iron chelates such as glycinate or biglycinate are used. Iron can also be elemental iron.

Micronutrient loaded lecithin vesicles may optionally comprise of one or more edge activators. Edge activators are preferably surfactants, which can be anionic, cationic preferably non-ionic and plasticizers, sodium taurocholate.

Preferred edge activators are selected from Solutol HS15, Tween 80, Tween 20, PEG 200, sodium taurocholate, PEG 400, Transcutol P, Span 80, sodium cholate, sodium deoxycholate, dipotassium glycyrrhizinate, sucrose laurate ester, L-595, a micelle-forming surfactant (octa oxyethylene laurate ester), PEG-8-L and their combinations. Most preferred edge activator is PEG 400 or PEG 200.

When edge activator is used, the ratio of micronutrient:lecithin:edge activator is from 1:0.5:0.1 to 1:5:2, preferably from 1:1:0.1 to 1:5:2 and most preferably from 1:1:0.1 to 1:2:2. In an embodiment as exemplified in examples 5A and 5 B, micronutrient:lecithin:edge activator ratio of 1:1:1 is used.

Under this aspect, a stabilizer and/or an antioxidant can be employed while preparing micronutrient loaded lecithin vesicles. In an embodiment under example 6, an ascorbic acid is added as a stabilizer/as an antioxidant. Alternatively, iron and amino acid complex can be used wherein amino acid complex/complexing agent or chelate/chelating agent serves as a stabilizer.

Under this aspect, the relative amounts of lecithin and edge activator determine particle size of the lecithin vesicles. Thus, it is possible to get particle size below 1 micron, below 500 nm and below 300 nm.

For example, table 5 provides various relative amounts of lecithin and edge activator wherein it is observed that when high amounts of edge activator is used and relatively low amounts of lecithin is used, it is possible to obtain vesicles of lower average particle size such as below 300, below 200 and below 100 nm. Similarly, when lecithin relative amount is higher than the amount of edge activator, vesicles of higher average particle size are obtained such as above 300 nm, above 400 nm and above 500 nm. The invention is not about a certain particle size, but it is about knowledge of various factors that can provide a desired particle size.

Second aspect of the present invention provides various further compositions comprising micronutrients loaded lipid vesicles/lecithin vesicles. These compositions include transdermal compositions such as i) peel-off gel, ii) film forming gel compositions, iii) lotions, iv) face cream composition v) clear transparent gel compositions vi) patch etc.

### Transdermal Peel Off Gel Compositions

The peel off gel compositions apart from micronutrient loaded elastic lipid vesicles/lecithin vesicles comprise of one or more of film former, gelling agent, thickening agent, preservatives, pH modifier and vehicle.

Preferred film former is one or more of Polyvinyl Alcohol, carbopol, HPMC K 4 M, K 100 M, Ethyl cellulose, Eudragit® RL100, Lutrol® F68 and PVP K-30, HPC, Eudragit RSPO, RLPO. The preferred film former is Polyvinyl Alcohol (PVA).

Gelling agents are one or more of Carbomers (carbopol), carboxy methyl cellulose, ethylcellulose, gelatin, hydroxyethyl cellulose, hydroxypropyl cellulose, magnesium aluminum silicate (Veegum®), methylcellulose, poloxamers (Pluronics®), polyvinyl alcohol, sodium alginate, tragacanth, and xanthan gum. The preferred Gelling agent is Carbopol (934 grade).

The transdermal peel-off gel compositions further may contain thickening agents, preservatives and pH modifiers.

Thickening agent is one or more of sodium carboxy methyl cellulose, PVP, phosphate esters, HPMC. The preferred Thickening agent is Sodium-carboxy methyl cellulose. The preferred Preservatives are Methyl paraben or Propyl paraben. The preferred pH Modifier is Triethanolamine.

Thickening agent or Viscosity builder can be one or more of sodium carboxy methyl cellulose, PVP, phosphate esters, HPMC.

The range in which each of the above ingredient is added is also crucial.

The preferred concentration of

1) gelling agent is from 0.01%-50%, preferably from 0.1 to 10% and most preferably from 0.5 to 5%

2) film Former is from 0.1-20%, preferably from 0.5 to 10% and most preferably from 1 to 8%

3) Thickening agent/viscosity builder is from 0.1-10%, preferably from 0.1 to 5% and most preferably from 0.1 to 4%

Also, nature of each of film former, gelling agent and thickening agent determines concentration. For example, if carbopol is used in very low concentration such as at or below 0.05%, it does not turn into semisolid. Thus, minimum concentration of carbopol to be used is 0.05 or above, preferably 0.1% and above and most preferably 0.25% and above.

For example, if polyvinyl alcohol is used in very low concentration such as at or below 0.5%, it does not turn into film. Thus, minimum concentration of polyvinyl alcohol to be used is 0.5% or above, preferably 2% and above and most preferably in the range of 3%-7%.

For example, if sodium carboxymethylcellulose is used in very low concentration, it does not turn into gel and if very high concentration is used, instead of peel off gel, a tacky gel composition is resulted. Thus optimum concentration of Sodium Carboxymethyl Cellulose is from 0.25%-4%.

Exemplary transdermal peel-off composition prepared in accordance with the present invention is provided under example 3.

Preparation of peel-off gel composition is done in two steps.

1) The first step involves preparation of micronutrients loaded lipid vesicles/lecithin vesicles and; 2) the second step is formulating micronutrients loaded lipid vesicles/lecithin vesicles into peel-off gel composition.

Micronutrients loaded lipid vesicles/lecithin vesicles are prepared as follows:

1. dissolve lipid/lecithin in a solvent;

2. dissolve micronutrient in the above solution,

3. evaporate the solution of step 2 to get thin film;

4. rehydrate the film to obtain vesicle.

Formulating above micronutrients loaded lipid vesicles/lecithin vesicles into peel-off gel composition is prepared as follows:

1. disperse polymer in water optionally with the help of mechanical stirrer;

2. allow polymer to swell or optionally stir until polymer swells;

3. disperse second polymer in above solution and allow polymer to swell or optionally stir gently until Polymer swells;

4. disperse and allow to swell third polymer such as sodium carboxymethylcellulose in water;

5. add micronutrient Loaded Lecithin Vesicles along with said polymer solution of step 3 to swelled third polymer such as NaCMC of step 4 under continuous stirring.

If needed, add triethanolamine to maintain pH and to achieve desired consistency of the formulation. Make up final volume with the purified water. After addition of all ingredients, stir continuously until a smooth dispersion is obtained.

The transdermal peel-off gel compositions are beneficial over conventional gel compositions having increased residence time, cosmetic appeal. Example 4 provides characterization of Peel-off gel.

Other preferred further compositions include film forming gel as exemplified in examples 7, 8 and 9. In an embodiment as exemplified in example 7, a film forming gel comprising ferrous sulphate loaded lecithin vesicles is provided. In an embodiment as exemplified in example 8, a film forming gel comprising folic acid/vitamin B9 loaded lecithin vesicles is provided. In yet another embodiment as exemplified in example 9, a film forming gel comprising three different types of lecithin vesicles is provided wherein each type of vesicle includes a different micronutrient such as either vitamin B12 or folic acid/vitamin B9 or ferrous sulphate. This film forming gel having above said micronutrients constitute a hematinic composition.

Under the second aspect, there is also provided a transdermal lotion composition. To prepare lotion composition, suitable lipids are selected for fatty phase. Suitable lipids include Cocoa butter, shea butter, cetostearyl alcohol, coconut buffer, Glyceryl monostearate, stearic acid etc. Aqueous phase contains i) humectant like glycerin, ii) alkalizer/soap forming agent such as potassium hydroxide and iii) pH modifier such as triethanolamine. The aqueous and fatty phases are mixed together and congealed to prepare a lotion. The micronutrients loaded one or more type of vesicles are added in the lotion and mixed to obtain lotion containing micronutrients loaded vesicles.

In an embodiment, a lotion having vitamin B12 loaded lecithin vesicles is prepared. Usually, the content of micronutrient loaded vesicles of lotion composition is from 0.1 to 20%, preferably from 0.5 to 10% and most preferably from 1 to 5%.

In yet another embodiment as exemplified in example 10, vitamin B12 loaded lecithin vesicles, folic acid loaded lecithin vesicles and ferrous sulphate loaded lecithin vesicles are mixed with the lotion to prepare a hematinic lotion composition. This composition has around 36% of micronutrients loaded vesicles. It is possible to incorporate up to around 50% of micronutrients loaded vesicles.

Further under this aspect, a transdermal patch can be prepared containing matrix type delivery system. In this, solvent casting method can be used. A single polymer or more than one polymer is dissolved in a suitable solvent to prepare solution. At least one Micronutrient loaded vesicles are added to this solution. The patch is prepared by casting the above mixture on release liner (Silicone coated polyester film) and by allowing it to dry. Additionally, the above solution may contain one or more permeation enhancers, one or more plasticizers and other suitable additives. The amounts of permeation enhancers and plasticizer may depend on total amount of polymer used. In an embodiment as exemplified in example 12, Polyethylene glycol 400 (30% w/w of total polymer) is used as plasticizer and propylene glycol (15% w/w of total polymer) is used as permeation enhancer.

Other examples of transdermal compositions include a face cream and clear transparent gel. In an embodiment as provided under example 11, preparation of clear transparent gel is provided.

Under the third aspect, the invention provides micronutrients loaded vesicles and the compositions thereof such that they provide rapid and improved permeation of micronutrients through skin into blood. By modifying the contents of vesicles and their further compositions, various types of release profiles can be achieved. Method for diffusion study is as provided under example 13 wherein In vitro permeation behavior of micronutrient loaded vesicles and plain micronutrient solution formulations were investigated using cellophane membrane. In this study, an In vitro drug release studies were performed using a Franz diffusion cell. The cellophane membrane with molecular weight cut off 1800 kilo Daltons1 was used for the determination of drug release. The phosphate buffer of pH 7.4 was used as a media.

Various permeation enhancers/edge activators are screened using an egg membrane. Micronutrients in different forms such as in solution form in different solvents including alcohol, water, DMSO etc. are screened through an egg membrane. Further, micronutrient loaded lecithin vesicles were tried. Some of the data of this study is as provided in Table 1 below. It indicates that micronutrients loaded lecithin vesicles exhibit better permeation than their solution with permeation enhancer DMSO.

TABLE 1
Permeation data for Vitamin B12

| Sr. | Permeation | B12 Lecithin | B12 solution in | B12 + |
|---|---|---|---|---|
| No | Parameters | Vesicle | water | DMSO |
| 1.1 | Amount Permeated | 2.933 | 2.080 | 2.278 |
| | (mg) after 6 hrs | | | |
| 2. | Lag time(min) | 7.02 | 37.62 | 23.82 |
| 3. | Enhancement Ratio | 1.41 | — | 1.088 |

In order to further improve the permeation, efforts were made to formulate flexible elastic vesicles by addition of various edge activators. Their effect on permeation was screened on egg membrane.

In further attempts to improve permeation of micronutrients, inventors prepared elastic lecithin vesicles. Thus, micronutrients loaded elastic lecithin vesicles are prepared using Vitamin B12, lecithin and various edge activators to impart elasticity to lecithin vesicles. Micronutrients loaded elastic lecithin vesicles using various different ratios from 1:1:0.1 to 1:2:2 of micronutrient, lecithin and edge activator are tried. Vitamin B12 loaded lecithin vesicles incorporating various edge activators in 1:1:0.5 ratio of Vitamin B12, lecithin and edge activators are prepared. Permeation of these lecithin vesicles using egg membrane are provided in table 2 below.

TABLE 2
Permeation data for screening of edge activators

| | | Ratio of | | % B12 permeated |
|---|---|---|---|---|
| Sr. | Edge | B12:Lecithin:Edge | Lag time | at end of 24 hrs |
| No | Activator | activator | in min | in μg |
| 1 | Tween 80 | 1:1:05 | 20 | 36.5 |
| 2 | Tween 20 | 1:1:05 | 15 | 33.2 |
| 3 | Transcutol P | 1:1:05 | 5 | 68 |
| 4 | PEG 400 | 1:1:05 | 5 | 84 |
| 5 | Span 80 | 1:1:05 | 30 | 23 |
| 6 | Solutol HS15 | 1:1:05 | 15 | 65 |

From above table, it reflects that PEG 400 provides the highest release. Thus, by using various edge activators and controlling their amounts, it is possible to obtain different release pattern of micronutrients.

Further, inventors of the present invention worked on best mode approach by conducting optimization trials using Factorial Design. In the factorial design for optimization, concentration of lecithin and concentration of edge activator are selected as independent variables and particle size, polydispersibility index and % cumulative drug release are dependent variables or responses.

Three levels of independent variables (factors X1 and X2) are chosen as provided in table 3 below and dependent variables (response Y1, Y2 and Y3) are obtained thereafter.

Thus, 3×3 factorial design is created as provided in table 4 below wherein three levels of lecithin and three levels of edge activator, PEG 400 are chosen and results obtained from such optimization trials are as provided under table 5 below.

TABLE 3
The Factors and Their Levels and corresponding
response used in Factorial Design

| | Independent | | | | |
|---|---|---|---|---|---|
| | variable/factor/Actual values | | | | |
| | X1 Concen- | X2 Concen- | | | |
| Levels | tration of | tration of | Dependent | | |
| (Code | Lecithin | PEG 400 | variable/Response | | |
| value) | (mg) | (mg) | Y1 | Y2 | Y3 |
| (−1) | 350 | 350 | Parti- | Poly | % Cumulative |
| (0) | 500 | 500 | cle | Disper- | drug release |
| (+1) | 1000 | 1000 | Size | sity | after 24 hours |
| | | | | Index | |

TABLE 4
Formulation trials using 3 X 3 factorial design

| RUN | Concentration of lecithin | Concentration of PEG 400 |
|---|---|---|
| 1. | +1 | +1 |
| 2. | +1 | −1 |
| 3. | +1 | 0 |
| 4. | −1 | +1 |
| 5. | −1 | 0 |
| 6. | −1 | −1 |
| 7. | 0 | +1 |
| 8. | 0 | 0 |
| 9. | 0 | −1 |

Each independent variable is kept at three levels viz. low level (−1), medium level (0) and high level (+1). Experimental trials are carried out at all nine possible combinations. The design layout and coded value of independent factor is shown in Table No 5. The factors are selected based on preliminary study. In all, 9 experimental runs are performed, each in triplicate and analyzed for the dependent variables.

The developed formulation is further optimized using 3² factorial design to get minimum particle size, polydispersity index and release criterion of NLT 85% in 24 hrs. The responses of various formulations are as shown in table No 5.

TABLE 5
Results from formulation optimization

| | Concen- | Concen- | | | | |
|---|---|---|---|---|---|---|
| | tration | tration | | | % Cumulative | |
| Formu- | of | of | Particle | | release in 24 | Std |
| lation | lecithin | PEG400 | size | PDI | hours | Dev |
| A1. | +1 | +1 | 272.6 | 0.317 | 92.9 ± 0.6 | 2.93 |
| A2. | +1 | −1 | 517.8 | 0.428 | 70.15 ± 0.92 | 2.429 |
| A3. | +1 | 0 | 297.1 | 0.212 | 66.74 ± 1.34 | 1.327 |
| A4. | −1 | +1 | 64.3 | 0.374 | 64.57 ± 1.34 | 0.795 |
| A5. | −1 | 0 | 351.6 | 0.238 | 84.34 | 0.436 |
| A6. | −1 | −1 | 313.4 | 0.459 | 95.54 ± 0.64 | 1.309 |
| A7. | 0 | +1 | 143.9 | 0.280 | 98.4 ± 1.23 | 1.704 |
| A8. | 0 | 0 | 236.4 | 0.267 | 71 ± 1.34 | 1.430 |
| A9. | 0 | −1 | 383.2 | 0.459 | 75 ± 1.23 | 1.3502 |

The relationship between the dependent (response) and independent variables (factor) is further elucidated using contour and response surface plots. 3D response surface plots give a representation of the variations in each response when the two factors are simultaneously changed from lower to higher level. It gives a three-dimensional curvature of the change in response at different factor levels. It also gives the variation in design points from the predicted response value.

### Response Y1

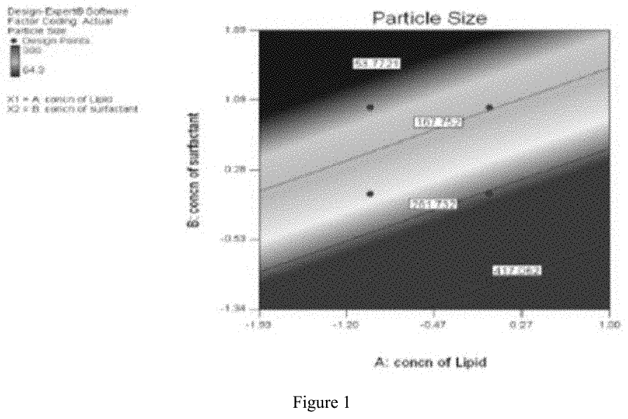

Response surface 3D plot illustrates the finding that as amount of PEG 400 is increased the particle size of the B12 loaded lecithin vesicles dispersion reduced. Same observations are also indicated by contour plot. This is provided in FIG. 1. Contour plot also shows that at high and medium level of PEG 400 the particle size is reduced. Also, Lecithin had the opposite effect as amount of Lecithin increased the particle size was increased, hence in order to get the optimum formulation the level of Lecithin should be kept to low or medium and PEG400 concentration high or medium.

### Response Y2

The particle size of vesicles significantly affects the physical stability of the formulation. If polydispersity index is high, there are high chances of particle aggregation and settling which will in turn affect the stability of the vesicle dispersion. Hence PDI should be kept minimum. PDI of B12 loaded vesicles varied from 0.223 to 0.63. From the data in Table 5, it is clear that PDI depends upon both the concentration of Lecithin and PEG 400 used in preparation and was found to be statistically significant at p<0.05.

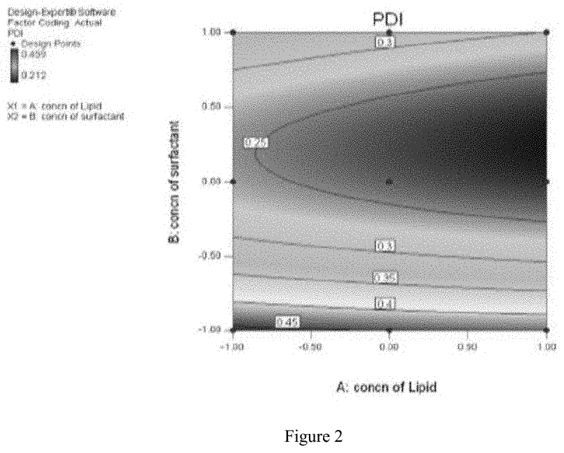

Response surface 3D plot illustrates the finding that as amount of PEG 400 was increased the PDI of the vesicles dispersion reduced. Same observations were also indicated by contour plot. Contour plot also shows that at high and medium level of PEG 400, the PDI is reduced. This is provided in FIG. 2. Also, Lecithin had the opposite effect as amount of Lecithin increased the PDI was increased, hence in order to get the optimum formulation the level of Lecithin should be kept to low or medium and PEG 400 concentration high or medium.

### Response Y3

The release profiles from vesicles can be modulated to obtain burst release, prolonged release (with no initial burst release) and different percentages of burst release followed by prolonged release. The release rate of the vesicles can be modified by proper choice of the Lecithin type, PEG400 or other edge activator concentration and production parameters. The % cumulative release for B12 Loaded Vesicles at end of 24 hrs varied from 64.5-98.4%. From the data in Table No 6, it is clear that % cumulative release depends upon the concentration of Lecithin used in preparation and was found to be statistically significant at p<0.05.

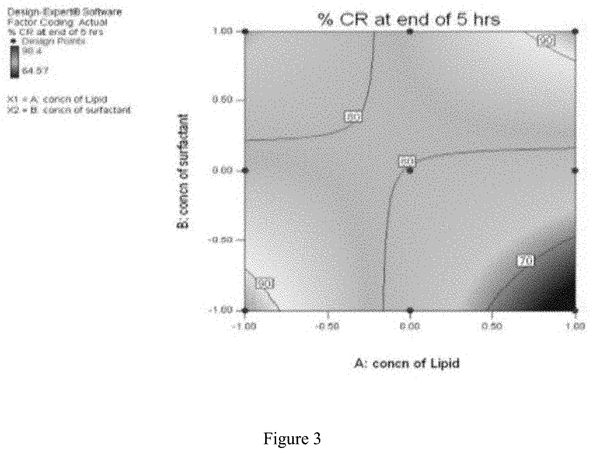

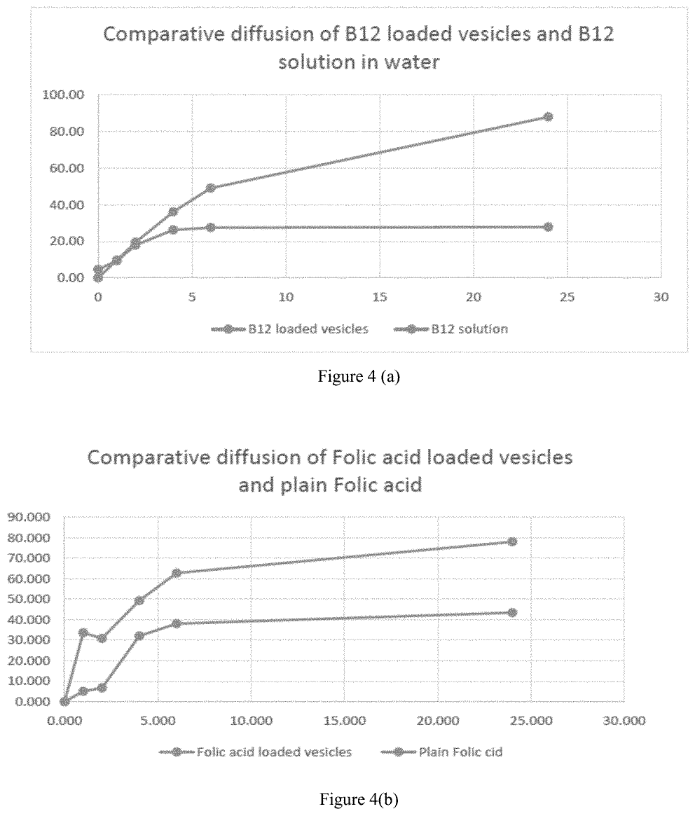

Response surface 3D plot illustrates the finding that as amount of PEG400 was increased the % cumulative release of the vesicles dispersion reduced. Same observations were also indicated by contour plot. This is provided in FIG. 3. Contour plot also shows that at high and medium level of PEG 400, the % cumulative release is increased. Also, Lecithin had the opposite effect as amount of Lecithin increased the % cumulative release was decreased, hence in order to get the optimum formulation the level of Lecithin should be kept to low or medium and PEG 400 concentration high or medium. The invention is not about a specific release pattern but it is about having knowledge of modulating release pattern which is a response obtainable by varying the independent variables.

Further, under this aspect, an In vivo study is performed to ascertain the transdermal delivery of micronutrients using further compositions comprising micronutrients loaded lecithin vesicles. Such study is provided under example 14. The test formulation for In vivo study is as provided under example 5B.

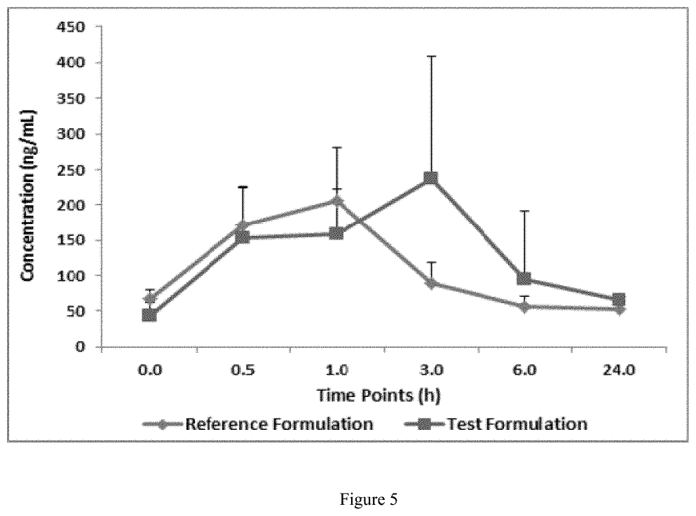

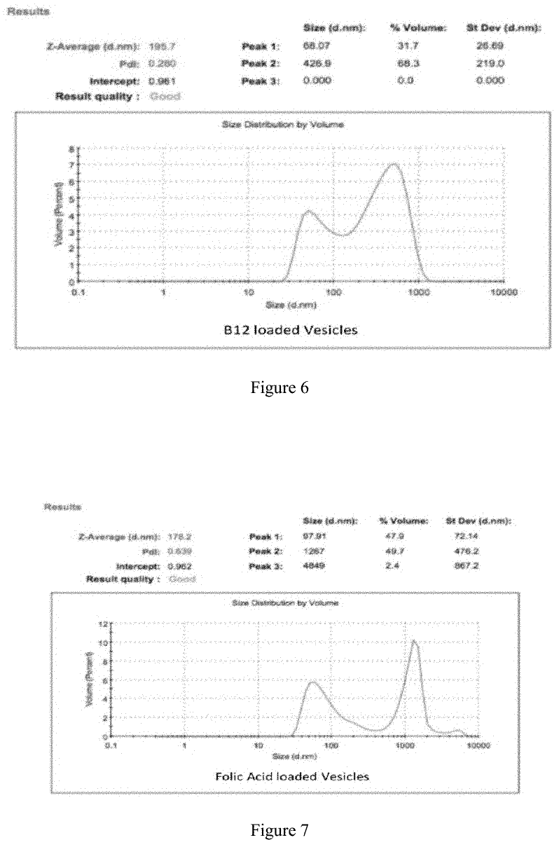

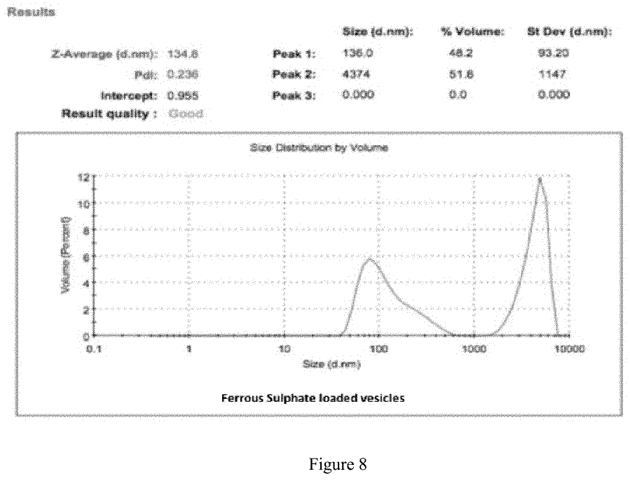

In vivo Data from this experiment is presented in FIG. 5.

The In vivo data of transdermal composition reflects increase in Tmax and AUC of transdermal composition as compared to the oral composition when the same dose is administered. The data as provided in table 11 indicates that the tmax is at least 1.5 times, preferably at least 2 times and most preferably at least 3 times of Tmax by oral composition. The AUC is preferably at least 1.1 times, more preferably at least 1.2 times and most preferably at least 1.3 times higher than AUC of oral composition when the same dose is administered.

Under fourth aspect, the invention provides process of preparing micronutrients loaded lipid vesicles/lecithin vesicles preferably phospholipid vesicles/lecithin vesicles and further processes to prepare their transdermal compositions that provide rapid and improved permeation through skin into blood.

### Process of Preparing Micronutrient Loaded Lecithin Vesicles

i) dissolving soya lecithin in a suitable solvent;

ii) dissolving micronutrient in above solution;

iii) optionally adding stabilizer/anti-oxidant to solution of step 2;

iv) optionally adding one or more edge activator to above solution and mixing;

v) evaporating solution to obtain thin film;

vi) rehydrating the film with water;

vii) obtaining nutrient loaded lecithin vesicles

wherein ratio of micronutrient to soya lecithin is from 1:0.5 to 1:5, more preferably from 1:0.5 to 1:2 and most preferably from 1:1 to 1:2 and wherein if edge activator is added in ratio/proportion of micronutrient:lecithin:edge activator is from 1:0.5:0.1 to 1:5:2, preferably from 1:1:0.1 to 1:5:2 most preferably from 1:1:0.1 to 1:2:2.

Further process of preparing micronutrient loaded lecithin vesicles comprises following steps:

i) dissolving soya lecithin in a suitable solvent;

ii) dissolving micronutrient in another solvent;

iii) optionally adding stabilizer/anti-oxidant to solution of step 2

iv) mixing solutions of steps 1 and 2 or 1 and 3 optionally under stirring;

v) optionally adding one or more edge activator to above solution and mixing;

vi) evaporating solution to obtain thin film;

vii) rehydrating the film with water;

viii) obtaining nutrient loaded lecithin vesicles

wherein ratio of micronutrient to soya lecithin is from 1:0.5 to 1:5, more preferably from 1:0.5 to 1:2 and most preferably from 1:1 to 1:2 and wherein if edge activator is added ratio/proportion of micronutrient:lecithin:edge activator is from 1:0.5:0.1 to 1:5:2, preferably from 1:1:0.1 to 1:5:2 most preferably from 1:1:0.1 to 1:2:2.

Preferably solvent used to dissolve lecithin is methanol. Preferably solvent used to dissolve vitamin B12 is also methanol. Solvent used to dissolve folic acid and ferrous sulphate are aqueous in nature preferably water or buffer such as phosphate buffer.

Process of preparing further compositions comprising micronutrient loaded lecithin vesicles is as follows:

### Film Forming Gel:

i) dispersing gelling agent such as Carbopol 934 in water and allowing it to swell;

ii) adding polymer such as Polyvinyl alcohol in Carbopol solution and allowing it to swell;

iii) preparing micronutrient/micronutrients loaded lecithin vesicles

iv) allowing sodium carboxymethyl cellulose to swell separately

v) adding Carbopol and PVA solution and micronutrient loaded lecithin vesicles to sodium CMC solution under stirring and optionally stirring

vi) adding triethanolamine to maintain pH and to achieve desired consistency

vii) optionally making up volume with water and further stirring

### Lotion

1) lipid phase is prepared by selecting suitable lipids such as cocao butter, shea butter, Glyceryl monostearate, stearic acid, cetosteryl alcohol and melting them based on the decreasing order of their melting points at 70 degrees Celsius

2) aqueous phase is separately prepared by heating water at 70° C. and adding in it appropriate quantity of potassium hydroxide, Triethalonamine and glycerin.

3) the aqueous and oily phase are mixed together under constant stirring and congealed.

4) water is added to make up the weight and desired consistency to obtain placebo lotion.

5) The above formed lotion is congealed to room temperature and appropriate vitamin E tocopherol, Folic acid Loaded Lecithin Vesicles, ferrous sulphate loaded lecithin vesicles and vitamin B12 loaded lecithin vesicles were added and mixed.

### Hematinic Clear Transparent Gel

1) dispersing gelling agent such as Carbopol 934 in water and allowing it to swell;

2) separately preparing folic acid Loaded Lecithin Vesicles, ferrous sulphate loaded lecithin vesicles and vitamin B12 loaded lecithin vesicles as explained previously;

3) separately preparing dispersion of sodium carboxy methyl cellulose and allowing it to swell;

4) adding swelled Carbopol dispersion and lecithin vesicles to swelled sodium carboxy methyl cellulose dispersion under stirring;

5) adding triethanolamine to adjust pH and to achieve desired consistency;

6) optionally making up volume with water and stirring to obtain smooth dispersion.

### Hematinic Patch

1) dissolving one or more polymers such as HPMC and polyvinyl alcohol in suitable solvent such as 1:1 of water and methanol;

2) separately preparing folic acid Loaded Lecithin Vesicles, ferrous sulphate loaded lecithin vesicles and vitamin B12 loaded lecithin vesicles as explained previously;

3) adding lecithin vesicles of step 2 to solution of step 1 and mixing until clear solution is obtained;

4) optionally adding plasticizer and permeation enhancer to above solution;

5) casting above solution on release liner of desired area and drying;

6) laminating the dried patches and cutting in desired sizes.

In a fifth aspect, the invention provides treatment of anemia by transdermal administration of micronutrients. Even though the oral route is the most common route for delivery, it is associated with several limitations. When a dietary supplement is swallowed, it is digested by stomach acids and then metabolized by the liver to the extent that only a small portion of the original dosage enters the bloodstream. In pregnant women, maternal metabolism is altered by hormones that mediate the redirecting of nutrients to the placenta and mammary gland as well as the transfer of nutrients to the developing infant. Absorption of some vitamins is dependent on presence of intrinsic factors; Oral bioavailability is majorly affected due to deficiency of these intrinsic factors.

Further, patients may not take oral supplements regularly due to various factors such as lack of knowledge, lack of awareness, forgetfulness, lack of resources etc. Iron supplements often interfere with GIT system and often produce nausea, loose motions or constipation. Once rural population experience such adverse effects, they stop taking the medication.

With respect to the present invention, it is possible for healthcare advisor associated with small villages and townships to physically administer such compositions and to be vigilant about the proper use of such compositions. Also, generally it is noted in various surveys that gel, cream and lotion compositions are more favored than oral compositions, more amongst the women population. The compositions like patch can be physically monitored. Hence, transdermal hematinic compositions which deliver and help in maintaining healthy levels of vitamin B12, folic acid, irons and other micronutrients are highly desirable.

Hematinic preparations are prepared by incorporating at least Vitamin B12, folic acid and iron preparations in the same transdermal composition. Examples 9-12 respectively provide hematinic film forming gel, lotion, clear transparent gel and transdermal patch, each containing at least 3 micronutrients loaded in lecithin vesicles. The dose of vitamin-B9 (folic acid) is from 80 mcg to 1000 mcg, dose of vitamin-B12 is from 4 mcg to 1200 mcg and dose of Iron is from 15 mg to 120 mg.

The present invention will now be demonstrated by reference to the following examples. It should be understood that these examples are disclosed solely by way of illustrating the invention and should not be taken in any way to limit the scope of the present invention.

### EXAMPLES

### Example 1

### Method of Preparation of Vesicles

1 gram of soya lecithin was dissolved in 25 ml of methanol to make stock solution of lecithin, 100 mg of Vitamin B12 was dissolved in 5 ml of lecithin stock solution to get a ratio of 1:2 of Vitamin B12:Lecithin. The above solution of methanol, lecithin and Vitamin B12 was evaporated on Rotovac [rotary evaporator] in a round bottom flask to get a thin film. The obtained film was rehydrated with 5 ml of water. These rehydrated vesicles were used for further permeation studies through membrane on franz diffusion cell.

### Example 2

### Preparation of the Transdermal Peel-Off Gel (B12 Loaded Lecithin Vesicles Incorporated in the Gel) Formulation

After optimisation of the polymer ingredients, peel-off gel formulation was prepared. Carbopol 934 dispersed in water with the help of mechanical stirrer and stirred continuously until Carbopol swelled. Polyvinyl alcohol (cold) was added in carbopol solution and stirred gently until PVA swells. B12 Loaded Lecithin Vesicles along with carbopol and PVA were added slowly in swelled NaCMC under continuous stirring. Triethanolamine was added to the obtained solution to maintain pH and to achieve desired consistency of the formulation. Final volume was made up with the purified water. After addition of whole ingredients, stirred continuously until a smooth dispersion obtained. Prepared formulation filled in lacquered plastic containers for further analysis.

### Example 3

TABLE 6
Transdermal peel-off gel composition

| | | | Optimised | |
|---|---|---|---|---|
| S. | | | concentration | |
| No | Ingredients | Category | (%) | |
| 1 | Vitamin B12 | Micronutrient | 1.0 | |
| | loaded lecithin | | | |
| | vesicles | | | |
| 2 | Polyvinyl Alcohol (PVA) | Film former | 6.0 | |
| 3 | Carbopol (934 grade) | Gelling agent | 0.5 | |
| 4 | Sodium carboxy | Thickening agent | 0.3 | |
| | methylcellulose | | | |
| 5 | Methyl paraben | Preservatives | 0.2 | |
| 6 | Propyl paraben | Preservatives | 0.02 | |
| 7 | Triethalonamine | pH Modifier | 1-2 | ml |
| 8 | Water | Vehicle | 100 | g |

### Example 4

### Pharmaceutical Characterisation of Peel-Off Gel

(i) pH

The pH value of transdermal peel off gel was determined by using digital pH meter. One gram of gel was dissolved in 100 ml distilled water and stored for two hours. The measurements of pH of the formulation were done in triplicate and average values calculated as given in table.

TABLE 7
Measurement of pH of the formulation

| Formulation/Vehicle | 1 | 2 | 3 | Average |
|---|---|---|---|---|
| Water | 7.6 | 7.5 | 7.6 | 7.5 ± 1 |
| Placebo peel-off gel | 7.5 | 7.5 | 7.5 | 7.0 ± 0.3 |
| B12 Lecithin Vesicle loaded peel-off gel | 7.1 | 7.5 | 7.6 | 7.1 ± 0.3 |

### (ii) Spreadability:

Spreadability of the peel-off gel was found to be 2.1±0.4 cm respectively.

TABLE 8
Spreadability of the Peel-off gel

| | | Weight | Length | Time |
|---|---|---|---|---|
| Formulation | Spreadability | (g) | (cm) | (sec) |
| Placebo Peel-off Gel | 0.22 | 2 | 2.5 | 11 |
| B12 Lecithin Vesicle | 0.27 | 2 | 2.5 | 11 |
| loaded peel-off gel | | | | |

(iii) Viscosity

TABLE 9
Viscosity of the formulations

| | | | VitaminB12 loaded |
|---|---|---|---|
| | | Placebo | lecithin Vesicle |
| S. No. | Parameters | Peel-off gel | containing Peel-off gel |
| 1 | Sample (g) | 1 | 1 |
| 2 | Speed (rpm) | 70 | 70 |
| 3 | Run | Triple | Triple |
| 4 | Run time (sec) | 60 | 60 |
| 5 | Temperature (° C.) | 30 ± 0.5 | 30 ± 0.5 |
| 6 | Shear rate (min−1) | 815.23 | 720.57 |
| 7 | Viscosity (cps) | 795 ± 25 | 770 ± 35 |

### Example 5A

500 miligram of soya lecithin was dissolved in 30 ml of methanol to make a stock solution of lecithin. Then 50 mg of Vitamin B12 was dissolved in 3 ml of lecithin stock solution to get a ratio of 1:1 [VitaminB12:lecithin]. To this solution 50 mg of polethylene glycol 200 (PEG 200) was added. Thus ratio of VitaminB12:lecithin:edge activator is 1:1:1.

The above solution of lecithin, Vitamin B12, polyethylene glycol 200 in methanol was evaporated on Rotovac [rotary evaporator] in a round bottom flask to get a thin film. The obtained film was rehydrated with 5 ml of water. These rehydrated vesicles were used for further permeation studies through membrane on franz diffusion cell.

### Example 5B

### Method of Preparation of a Film Forming Gel Comprising Vitamin B12 Loaded Lecithin Vesicles Using Gelling and Film Forming Agent as Follows

Appropriate quantity of Carbopol 934 was dispersed in water with the help of mechanical stirrer and stirred continuously until Carbopol swelled. Polyvinyl alcohol was added in Carbopol solution and stirred gently until Poly vinyl alcohol swelled. Appropriate quantity of vitamin B12 loaded lecithin vesicles containing PEG 200 as provided under example 5A were added slowly in swelled sodium carboxy methyl cellulose under continuous stirring. Triethalonamine was added to the above mixture solution to maintain pH and to achieve desired consistency of the formulation. Final volume was made up with the purified water. After addition of whole ingredients, stirred continuously until a smooth dispersion obtained. The test formulation contains 1% of lecithin vesicles. A 3 g gel thus contains 0.03 g lecithin vesicles=30 mg of lecithin vesicles. The lecithin vesicles are made up of 1:1:1 of vitamin B12:lecithin:PEG 200. Thus 30 mg of lecithin vesicles contained 10 mg of vitamin B12.

### Example 5C

### Preparation of Folic Acid Loaded Vesicles

50 miligram of folic acid was dissolved in 10 ml of pH 7.4 phosphate buffer to make a stock solution of folic acid 5 mg/ml. 500 milligram of soya lecithin was dissolved in 30 ml of methanol to make stock solution of lecithin. Lecithin stock solution and folic acid stock solution were then mixed on a magnetic stirrer with addition of Polyethylene glycol 200. The ratio of soya lecithin:Folic Acid:Polyethylene glycol 200 was 1:1:1. The above solution of folic acid stock, polyethylene glycol lecithin stock was evaporated on Rotovac [rotary evaporator] in a round bottom flask to get a thin film. The obtained film was rehydrated with 5 ml of water 45 degree Celsius on rotary evaporator. These rehydrated vesicles were used for further permeation studies through membrane on franz diffusion cell.

### Example 6

### Preparation of Ferrous Sulphate Loaded Vesicles

Aqueous solution of ferrous Sulphate was made to get a stock solution of 30 mg/ml. Ascorbic acid was added to the above solution to prevent oxidation of ferrous ions. the ratio of Ferrous sulphate:Ascorbic acid was kept 10:1. 500 milligram of soya lecithin was dissolved in 30 ml of methanol to make stock solution of lecithin. Lecithin stock solution and Ferrous sulphate stock solution were then mixed on a magnetic stirrer with addition of Polyethylene glycol 200. The ratio of soya lecithin:Folic Acid:Polyethylene glycol 200 was 1:1:1. The above solution of folic acid stock, polyethylene glycol lecithin stock was evaporated on Rotovac [rotary evaporator] in a round bottom flask to get a thin film. The obtained film was rehydrated with 5 ml of water at 45 degrees Celsius on rotary evaporator. These rehydrated vesicles were used for further permeation studies through membrane on Franz diffusion cell.

### Example 7

### Method of Preparation of Ferrous Sulphate Vesicles Loaded Film Forming Gel

Carbopol 934 dispersed in water with the help of mechanical stirrer and stirred continuously until Carbopol swelled. Polyvinyl alcohol was added in Carbopol solution and stirred gently until Poly vinyl alcohol swelled. Ferrous sulphate Loaded Lecithin Vesicles along with Carbopol and PVA were added slowly in swelled sodium carboxy methyl cellulose under continuous stirring. Triethalonamine was added to the obtained solution to maintain pH and to achieve desired consistency of the formulation. Final volume was made up with the purified water. After addition of whole ingredients, stirred continuously until a smooth dispersion obtained. Prepared formulation filled in lacquered plastic containers for further analysis. Ferrous sulphate containing film forming gel was stable on storage.

### Example 8

### Method of Preparation of Folic Acid Vesicles Loaded in Film Forming Gel

Appropriate quantity of Carbopol 934 was dispersed in water with the help of mechanical stirrer and stirred continuously until Carbopol swelled. Polyvinyl alcohol was added in Carbopol solution and stirred gently until Poly vinyl alcohol swelled. Appropriate quantity of Folic acid Loaded Lecithin Vesicles along with Carbopol and PVA were added slowly in swelled sodium carboxy methyl cellulose under continuous stirring. Triethalonamine was added to the above mixture solution to maintain pH and to achieve desired consistency of the formulation. Final volume was made up with the purified water. After addition of whole ingredients, stirred continuously until a smooth dispersion obtained. Prepared formulation filled in lacquered plastic containers for further analysis. Folic acid containing film forming gel was stable on storage

### Example 9

### Method of Preparation of Transdermal Hematinic Film Forming Gel

Appropriate quantity of Carbopol 934 was dispersed in water with the help of mechanical stirrer and stirred continuously until Carbopol swelled. Polyvinyl alcohol was added in Carbopol solution and stirred gently until Poly vinyl alcohol swelled. Appropriate quantity of Folic acid Loaded Lecithin Vesicles, ferrous sulphate loaded lecithin vesicles and vitamin B12 loaded lecithin vesicles along with Carbopol and PVA were added slowly in swelled sodium carboxy methyl cellulose under continuous stirring. Triethalonamine was added to the above mixture solution to maintain pH and to achieve desired consistency of the formulation. Final volume was made up with the purified water. After addition of whole ingredients, stirred continuously until a smooth dispersion obtained. Prepared formulation was filled in lacquered plastic containers for further analysis. The transdermal hematinic film forming gel was stable on storage.

### Example 10

### Method of Preparation of Transdermal Hematinic Lotion

TABLE 10
Composition of transdermal hematinic lotion

| Ingredient for transdermal hematinic lotion | Quantity in % w/w |
|---|---|
| Cocoa butter | 12 |
| Shea butter | 2 |
| Glyceryl monostearate | 6 |
| Stearic Acid | 3 |
| Cetosteryl alcohol | 3 |
| Tri ethanol amine | 1.5 |
| Potassium hydroxide | 1 |
| Glycerine | 15 |
| Water | Qs to 100% |

Appropriate quantity of cocao butter, shea butter, Glyceryl monostearate, stearic acid, cetosteryl alcohol was heated in a vessel based on the decreasing order of melting point at 70 degrees Celsius. This constituted of the lipid phase. In a separate vessel water was heated to 70 degrees Celsius with addition of appropriate quantity of potassium hydroxide, Triethalonamine and glycerin. The above solution constituted the aqueous phase.

The aqueous and oily phase were mixed together under constant stirring and congealed. water was added to make up the weight and desired consistency. The above formed lotion was congealed to room temperature and appropriate vitamin E tocopherol, Folic acid Loaded Lecithin Vesicles, ferrous sulphate loaded lecithin vesicles and vitamin B12 loaded lecithin vesicles were added and mixed. Around 160 g of lotion is mixed with 90 g of vesicles and water is added to make up 100% w/w. The 90 g vesicles contain around 30 g of vitamin B12 loaded lecithin vesicles, around 30 g of folic acid loaded lecithin vesicles and around 30 g of ferrous sulphate loaded lecithin vesicles.

Prepared formulation was filled in lacquered plastic containers for further analysis. The transdermal hematinic lotion was stable on storage.

The dose is calculated as follows:

Around 250 g lotion contains 30 g of each type of lecithin vesicles. Lecithin vesicles contain 1:1:1 of micronutrient:lecithin:edge activator. Thus 30 g of lecithin vesicles contain around 10 g of vitamin B12/folic acid/ferrous sulphate.

The amount of lotion to be applied is from 1 g to 10 g.

Thus 1 g contains 0.04 g of each of vitamin B12, folic acid and ferrous sulphate.

Thus 10 g contains 0.4 g of each of vitamin B12, folic acid and ferrous sulphate.

Alternatively, by mixing different amounts of micronutrients loaded lecithin vesicles with placebo lotion, different doses of micronutrients can be achieved.

### Example 11

### Method of Preparation of Transdermal Hematinic Clear Transparent Gel

Appropriate quantity of Carbopol 934 was dispersed in water with the help of mechanical stirrer and stirred continuously until Carbopol swelled. Appropriate quantity of Folic acid Loaded Lecithin Vesicles, ferrous sulphate loaded lecithin vesicles and vitamin B12 loaded lecithin vesicles along with Carbopol were added slowly in swelled sodium carboxy methyl cellulose under continuous stirring. Triethalonamine was added to the above mixture solution to maintain pH and to achieve desired consistency of the formulation. Final volume was made up with the purified water. After addition of whole ingredients, stirred continuously until a smooth dispersion obtained. Prepared formulation was filled in lacquered plastic containers for further analysis. The transdermal hematinic film forming gel was stable on storage.

### Example 12

### Method of Preparation of Transdermal Hematinic Patch

Micronutrient vesicle loaded matrix-type transdermal patches were prepared by using solvent casting method. A petri dish with a total area of 44.15 cm2 was used. HPMC K100 and Poly vinyl alcohol were accurately weighed and dissolved in 10 mL of water, methanol (1:1) solution and kept aside to form clear solution. Accurately weighed Folic acid Loaded Lecithin Vesicles, ferrous sulphate loaded lecithin vesicles and vitamin B12 loaded lecithin vesicles were dissolved in the above solution and mixed until clear solution was obtained. Polyethylene glycol 400 (30% w/w of total polymer) was used as plasticizer and propylene glycol (15% w/w of total polymer) was used as permeation enhancer. The patch was prepared by casting the above mixture on release liner (Silicone coated polyester film) and allowed to dry at room temperature overnight. An inverted funnel was placed over the petri dish to prevent fast evaporation of the solvent. The dried patches were laminated and cut into 9.61 cm2 area and stored in polyethylene bag at 40° C./75% R.H. till the further evaluation.

### Example 13

### Method for Diffusion Study

In vitro permeation behavior of micronutrient loaded vesicles and plain micronutrient solution formulations was investigated using cellophane membrane (Molecular weight cut of 12000-14000, HI Media Ltd, Mumbai, India). The vertical type of Franz Diffusion cell (DBK Diffusion cell apparatus) of appropriate amount of the vesicles was applied on 3.14 cm 2 area of the surface of cellophane membrane fixed to the lower end of donor compartment. The volume of the receptor fluid was kept 24 ml. The temperature of the receptor buffer (Phosphate buffer pH7.4) was maintained at 37±0.5° C. and stirred continuously at 100 Aliquots of 2.0 ml were withdrawn and analyzed for the drug content after suitable dilutions by spectrophotometric method. The volume of buffer was replaced with the same volume of fresh buffer after each sampling. The cumulative amount permeated across the cellophane membrane was calculated and plotted against time.

### Example 14

### Method for Animal Study

Animals were randomized on the last day of acclimatization using stratification method of randomization and were grouped accordingly such that the mean body weights did not exceeded ±20% among the groups. 50 mg of the reference standard Cynacobalamine (vitamin B12) was weighed and transferred to a 15 ml vial. The compound was dissolved in 5 ml of distilled water and then made up to 10 ml with distilled water to make a solution of 5 mg/ml concentration. For group 1 (G1), a dose volume of 2 ml formulation containing 10 mg of reference standard was administered to each rat irrespective of the body weight. Test formulation is as provided in example 5B. Test formulation was used as such as the formulation is in gel form for dermal application. 3 gm of the gel formulation containing 10 mg of test cynacobalamine is a test formulation.

One day prior to the dermal application of test item formulation for group 2 (G2) rats, the hair from the back of each rat was removed using hair removal cream and washed with saline. On the day of experiment, the shaved area was wiped with 70% isopropyl alcohol and dried. The area of application (3.5 cm×7.0 cm) was marked and the 3 gm of the gel formulation containing 10 mg of test cynacobalamine (vitamin B12) in lecithin vesicles was applied evenly. Immediately after application, the area was covered with non-absorbable cotton and was secured with adhesive tape. The formulation was withheld in position for a period of 24 hrs. the animals were observed for clinical sign of mortality and body weight.

Blood was collected through retro orbital plexus using capillary at specified time points i.e. 0.5 hr, 1.0 hr, 3.0 hr, 6.0 hr & 24.0 hrs post-dose. To collect the serum sample, the tubes were kept for 30 min. at room temperature and allowed for coagulation and then separated by centrifugation the tubes at 3000 rpm for 10 min at 4° C.

TABLE 11
Comparison of In vivo data for oral and transdermal route

| Parameter | Oral | Transdermal |
|---|---|---|
| Dose (mg) | 10 | 10 |
| Tmax (hr) | 1 | 3.0 |
| Cmax (ng/ml) | 205.67 | 236.18 ng/ml |
| AUC (0-∞) (ng/mL*h) | 2775.15 | 3691.31 |
| F (%) | — | 67.96 |

ⁱ B. Godin, E. Touitou, Transdermal skin delivery: Predictions for humans from in vivo, ex vivo and animal models, Advanced Drug Delivery Reviews. 59(11), 30 Sep. 2007, 1152-1161.
ⁱⁱ K. Harada, T. Murakami, E. Kawasaki, Y. Higashi, S. Yamamoto, N. Yata, In-vitro permeability to salicylic acid of human, rodent, and shed snake skin, J. Pharm. Pharmacol. 45 (1993) 414-418.

1. Topically applied Pharmaceutical or nutraceutical composition comprising at least one micronutrient loaded lecithin vesicles for transdermal use.

2. Topically applied Pharmaceutical or nutraceutical composition according to the claim 1 further comprising one or more edge activators.

3. Topically applied Pharmaceutical or nutraceutical composition according to the claim 2 wherein the edge activator is selected from the group consisting of one or more of Solutol HS15, PEG 200, sodium taurocholate, PEG 400, Transcutol P, sodium cholate, sodium deoxycholate, Span 80, Tween 20, Tween 80, dipotassium glycyrrhizinate, sucrose laurate ester, sodium lauryl ether sulphate, a micelle-forming surfactant (octa oxyethylene laurate ester), PEG-8-L and combinations thereof.

4. Topically applied Pharmaceutical or nutraceutical composition according to the claim 2 wherein the edge activator is PEG 200 or PEG 400.

5. Topically applied Pharmaceutical or nutraceutical composition comprising at least one micronutrient loaded lecithin vesicles for transdermal use wherein the topical composition is in any one form selected from the group consisting of peel off gel, film forming gel, transdermal patch, lotion, face cream and clear transparent gel.

6. Topically applied Pharmaceutical or nutraceutical composition according to the claim 1 or claim 5 wherein micronutrients include one or more of Vitamin-A, Vitamin-D, Vitamin-E, Vitamin-C, Vitamin-B1, Vitamin-B2, Vitamin-B3, Vitamin-B6, Vitamin-B9 (folic acid), Vitamin-B12, Zinc, Copper, Selenium, Iodine, Iron, and salts, complexes or chelates thereof.

7. Topically applied Pharmaceutical or nutraceutical composition according to the claim 6 wherein micronutrient include one or more of vitamin B9 (folic acid), vitamin B12, Iron, and salts, complexes or chelates thereof.

8. Topically applied Pharmaceutical or nutraceutical composition comprising micronutrient loaded lecithin vesicles for transdermal use wherein micronutrient is one or more of vitamin B9 (folic acid), vitamin B12, and Iron and wherein dose of vitamin-B9 (folic acid) is from 80 mcg to 1000 mcg, dose of vitamin-B12 is from 4 mcg to 1200 mcg and dose of Iron is from 15 mg to 120 mg.

9. Topical lotion comprising micronutrient loaded lecithin vesicles for transdermal use wherein micronutrient is one or more of vitamin B9 (folic acid), vitamin B12, Iron, and salts, complexes or chelates thereof.

10. Topical peel off gel composition comprising micronutrient loaded lecithin vesicles for transdermal use wherein micronutrient is one or more of vitamin B9 (folic acid), vitamin B12, Iron, and salts, complexes or chelates thereof.

11. Topical film forming gel composition comprising micronutrient loaded lecithin vesicles for transdermal use wherein micronutrient is one or more of vitamin B9 (folic acid), vitamin B12, Iron, and salts, complexes or chelates thereof.

12. Topical patch comprising micronutrient loaded lecithin vesicles for transdermal use wherein micronutrient is one or more of vitamin B9 (folic acid), vitamin B12, Iron, and salts, complexes or chelates thereof.

13. Face cream comprising micronutrient loaded lecithin vesicles for transdermal use wherein micronutrient is one or more of vitamin B9 (folic acid), vitamin B12, Iron, and salts, complexes or chelates thereof.

14. Topical clear transparent gel composition comprising micronutrient loaded lecithin vesicles for transdermal use wherein micronutrient is one or more of vitamin B9 (folic acid), vitamin B12, Iron, and salts, complexes or chelates thereof.

15. Micronutrient loaded lecithin vesicles comprising micronutrient and lecithin in a ratio from 1:0.5 to 1:5, more preferably from 1:0.5 to 1:2 and most preferably from 1:1 to 1:2.

16. Micronutrient loaded lecithin vesicles of claim 15 further comprising one or more of edge activator wherein ratio of micronutrient:lecithin:edge activator is from 1:0.5:0.1 to 1:5:2, preferably from 1:1:0.1 to 1:5:2 most preferably from 1:1:0.1 to 1:2:2.

17. Micronutrient loaded lecithin vesicles of claim 16 wherein the edge activator is selected from the group consisting of one or more of Solutol HS15, Tween 80, Tween 20, PEG 200, sodium taurocholate, PEG 400, Transcutol P, sodium cholate, sodium deoxycholate, Span 80, and dipotassium glycyrrhizinate, sucrose laurate ester, sodium lauryl ether sulphate, a micelle-forming surfactant (octa oxyethylene laurate ester), PEG-8-L and combinations thereof.

18. Process of preparing micronutrient loaded lecithin vesicles comprising
i) dissolving soya lecithin in a suitable solvent;
ii) dissolving micronutrient in above solution;
iii) optionally adding stabilizer/anti-oxidant to solution of step 2;
iv) optionally adding one or more edge activator to above solution and mixing;
v) evaporating solution to obtain thin film;
vi) rehydrating the film with water;
vii) obtaining nutrient loaded lecithin vesicles
wherein ratio of micronutrient to soya lecithin is from 1:0.5 to 1:5, more preferably from 1:0.5 to 1:2 and most preferably from 1:1 to 1:2 and wherein if edge activator is added ratio of micronutrient:lecithin:edge activator is from 1:0.5:0.1 to 1:5:2, preferably from 1:1:0.1 to 1:5:2 most preferably from 1:1:0.1 to 1:2:2.

19. Process of preparing micronutrient loaded lecithin vesicles comprising
i) dissolving soya lecithin in a suitable solvent;
ii) dissolving micronutrient in another solvent;
iii) optionally adding stabilizer/anti-oxidant to solution of step 2
iv) mixing solutions of steps 1 and 2 or 1 and 3 optionally under stirring;
v) optionally adding one or more edge activator to above solution and mixing;
vi) evaporating solution to obtain thin film;
vii) rehydrating the film with water;
viii) obtaining nutrient loaded lecithin vesicles
wherein ratio of micronutrient to soya lecithin is from 1:0.5 to 1:5, more preferably from 1:0.5 to 1:2 and most preferably from 1:1 to 1:2 and wherein if edge activator is added, ratio of micronutrient:lecithin:edge activator is from 1:0.5:0.1 to 1:5:2, preferably from 1:1:0.1 to 1:5:2 most preferably from 1:1:0.1 to 1:2:2.

20. Process of preparing micronutrient loaded lecithin vesicles containing composition comprising
i) dispersing gelling agent in water and allowing it to swell;
ii) adding polymer in gelling agent solution and allowing it to swell;
iii) preparing micronutrient/micronutrients loaded lecithin vesicles;
iv) disperse thickening agent separately allowing it to swell;
v) adding gelling agent and polymer solution and micronutrient loaded lecithin vesicles to thickening agent solution under stirring and optionally stirring;
vi) adding pH modifier/triethanolamine to maintain pH and to achieve desired consistency;
vii) optionally making up volume with water and further stirring
wherein the composition is a film forming gel.

21. Process of preparing micronutrient loaded lecithin vesicles containing composition comprising
i) dispersing gelling agent in water and optionally stirring until it swells;
ii) adding polymer in gelling agent dispersion and optionally stirring until polymer swells;
iii) separately preparing dispersion/solution of thickening agent in water and allowing it to swell;
iv) adding vitamin B12 Loaded Lecithin Vesicles along with gelling agent and polymer solution/dispersion in swelled thickening agent under continuous stirring;
v) adding pH modifier/Triethanolamine to maintain pH and to achieve desired consistency
vi) optionally making final volume with purified water and stirring until a smooth dispersion is obtained;
wherein the composition is a peel off gel.

22. Process of preparing micronutrient loaded lecithin vesicles containing composition comprising
i) preparing lipid phase by selecting suitable lipids and melting them based on the decreasing order of their melting points at around 70 degrees Celsius;
ii) preparing aqueous phase by heating water at 70° C. and adding in it appropriate quantity of humectant, alkalizer and pH modifier;
iii) mixing the aqueous and oily phase under constant stirring and congealing;
iv) adding water to make up weight and to obtain placebo lotion;
v) congealing the above formed lotion at room temperature and adding at least one or more lecithin vesicles selected from vitamin B12 loaded lecithin vesicles, Folic acid Loaded Lecithin Vesicles and iron/iron salt/iron chelate loaded lecithin vesicles and mixing;
wherein the composition is a lotion or a hematinic lotion.

23. Process of preparing micronutrient loaded lecithin vesicles containing composition comprising
i) dispersing gelling agent in water and allowing it to swell;
ii) separately preparing one or more of folic acid Loaded Lecithin Vesicles, iron/iron salt/iron chelate loaded lecithin vesicles and vitamin B12 loaded lecithin vesicles;
iii) separately preparing dispersion of thickening agent and allowing it to swell;
iv) adding swelled gelling agent dispersion and one or more lecithin vesicles to swelled thickening agent dispersion under stirring;
v) adding pH modifier to adjust pH and to achieve desired consistency;
vi) optionally making up volume with water and stirring to obtain smooth dispersion
wherein the composition is a clear transparent gel or a hematinic clear transparent gel.

24. Process of preparing micronutrient loaded lecithin vesicles containing composition comprising
i) dissolving one or more polymers in suitable solvent;
ii) separately preparing one or more of vitamin B12 loaded lecithin vesicles, folic acid Loaded Lecithin Vesicles and iron/iron salt/iron chelate loaded lecithin vesicles;
iii) adding lecithin vesicles of step 2 to solution of step 1 and mixing until clear solution is obtained;
iv) optionally adding plasticizer and permeation enhancer to above solution;
v) casting above solution on release liner of desired area and drying;
vi) laminating the dried patches and cutting in desired sizes;
wherein the composition is a transdermal patch or a transdermal hematinic patch.

25. Topically applied Pharmaceutical or nutraceutical composition comprising at least one micronutrient loaded lecithin vesicles for transdermal use having Tmax in an animal at least 1.5 times, preferably at least 2 times and most preferably at least 3 times of Tmax in the same animal of an oral composition having same micronutrient in same dose.

26. Topically applied Pharmaceutical or nutraceutical composition comprising at least one micronutrient loaded lecithin vesicles for transdermal use having AUC in an animal at least 1.1 times, preferably at least 1.2 times and most preferably at least 1.3 times of AUC in the same animal of an oral composition having same micronutrient in same dose.

27. Topically applied Pharmaceutical or nutraceutical composition according to any preceding claims wherein micronutrient is selected from the group consisting of Vitamin B12, folic acid and iron salt/chelate or elemental iron.
